(19) United States
(12) Patent Application Publication
(10) Pub. No.: US 2004/0220628 A1
(43) Pub. Date: Nov. 4, 2004

(54) Subcutaneous defibrillation timing correlated with induced skeletal muscle contraction

(76) Inventor: Darrell Orvin Wagner, Isanti, MN (US)

Correspondence Address: Crawford Maunu PLLC, Suite 390, 1270 Northland Drive, St. Paul, MN, 55120, US

(21) Appl. No.: 10/818,188

(22) Filed: Apr. 5, 2004

Related U.S. Application Data
(60) Provisional application No. 60/462,272, filed on Apr. 11, 2003.

(51) Int. Cl. A61N 1/39

(52) U.S. Cl. 607/5

(57) ABSTRACT

Methods and systems for defibrillation therapy involve delivering a pre-shock waveform to cause contraction of skeletal musculature in the patient's thorax before delivering a defibrillation waveform. A shock is delivered to the patient's heart during contraction of the skeletal musculature for a reduction in defibrillation threshold. The pre-shock waveform is sufficient in energy to cause one or both of deflation of the patient's lungs and muscle fiber shortening of the skeletal musculature. A delay interval may be initiated relative to delivery of the pre-shock waveform, wherein the defibrillation waveform is delivered following expiration of the delay interval. Motion of the patient's thorax and/or expiration of the patient's lungs may be detected, responsive to the pre-shock waveform. The defibrillation waveform may be delivered in coordination with the detected parameter, such as in relation to detection of a peak in the thoracic motion or minimum in transthoracic impedance.

### RELATED APPLICATIONS

This application claims the benefit of Provisional Patent Application Serial No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to implantable medical devices and, more particularly, to subcutaneous cardiac sensing and/or stimulation devices employing subcutaneous defibrillation correlated with skeletal muscle stimulation.

### BACKGROUND OF THE INVENTION

The healthy heart produces regular, synchronized contractions. Rhythmic contractions of the heart are normally controlled by the sinoatrial (SA) node, which is a group of specialized cells located in the upper right atrium. The SA node is the normal pacemaker of the heart, typically initiating 60-100 heartbeats per minute. When the SA node is pacing the heart normally, the heart is said to be in normal sinus rhythm (NSR).

If the heart's electrical activity becomes uncoordinated or irregular, the heart is denoted to be arrhythmic. Cardiac arrhythmia impairs cardiac efficiency and may be a potential life-threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria may also adversely affect the ventricular rate.

Ventricular tachycardia occurs, for example, when electrical activity arises in the ventricular myocardium at a rate more rapid than the normal sinus rhythm. Ventricular tachycardia may quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Typical Implantable cardioverter/defibrillators (ICDs) include one or more endocardial leads to which at least one defibrillation electrode is connected. Such ICDs are capable of delivering high-energy shocks to the heart, interrupting the ventricular tachyarrhythmia or ventricular fibrillation, and allowing the heart to resume normal sinus rhythm. ICDs may also include pacing functionality.

Although ICDs are very effective at preventing Sudden Cardiac Death (SCD), most people at risk of SCD are not provided with implantable defibrillators. Primary reasons for this unfortunate reality include the limited number of physicians qualified to perform transvenous lead/electrode implantation, a limited number of surgical facilities adequately equipped to accommodate such cardiac procedures, and a limited number of the at-risk patient population that may safely undergo the required endocardial or epicardial lead/electrode implant procedure.

### SUMMARY OF THE INVENTION

The present invention is directed to subcutaneous defibrillation systems and methods that provide cardiac shock delivery coordinated with skeletal muscle stimulation. Embodiments of the present invention are directed to subcutaneous cardiac monitoring and/or stimulation methods and systems that detect and/or treat cardiac activity or arrhythmias.

According to one embodiment of the invention, a method of delivering a subcutaneous defibrillation therapy involves delivering, from a subcutaneous non-intrathoracic location relative to a patient's heart, a pre-shock waveform sufficient in energy to cause contraction of skeletal musculature in the patient's thorax but insufficient in energy to defibrillate the patient's heart. A defibrillation waveform is delivered to the patient's heart during contraction of the skeletal musculature. The pre-shock waveform is sufficient in energy to cause one or both of deflation of the patient's lungs and muscle fiber shortening of the skeletal musculature.

Displacement of the patient's heart relative to the electrodes due to the pre-shock achieves an increased defibrillation current density in the patient's heart relative to a defibrillation current density in the patient's heart achievable in the absence of displacement of the patient's heart. Contraction of the skeletal musculature may reduce a defibrillation threshold relative to a defibrillation threshold associated with delivery of the defibrillation waveform in the absence of the skeletal musculature contraction. A delay interval may be initiated relative to delivery of the pre-shock waveform, wherein the defibrillation waveform is delivered following expiration of the delay interval. The delay interval may have a pre-established duration. For example, the delay interval may have a duration equal to or less than about 2 seconds, equal to or less than about 200 milliseconds or equal to about 100 milliseconds.

Pre-shock waveforms may have, for example, a pulse width equal to or less than about 2 milliseconds and/or equal to or less than about 20% of that of the defibrillation waveform. Pre-shock waveforms may have, for example, an initial amplitude greater than that of the defibrillation waveform or an initial amplitude less than that of the defibrillation waveform. Pre-shock waveforms may be, for example, monophasic waveforms or multiphasic waveforms, such as a biphasic, truncated exponential waveform. The pre-shock waveform may have an energy level, a first phase, and a second phase, where the energy level of one or both of the first phase and the second phase of the defibrillation waveform is reduced by an amount of energy corresponding to the energy level of the pre-shock waveform. The pre-shock waveform may be delivered using a first vector, and the defibrillation waveform may be delivered using the first vector or a second vector differing from the first vector.

Motion of the patient's thorax may be detected, responsive to the pre-shock waveform. The defibrillation waveform may be delivered in coordination with the detected motion, such as in relation to detection of a peak in the thoracic motion, and/or using a pre-determined delay interval between delivery of the defibrillation and pre-shock waveforms. In another embodiment, expiration of the patient's lungs may be detected in response to the pre-shock waveform using transthoracic impedance, and the defibrillation waveform may be delivered in coordination with the detected lung expiration, such as by detection of a minimum in the transthoracic impedance.

Embodiments of the present invention are also directed to a system for delivering a subcutaneous defibrillation therapy. The system may have a housing configured for subcutaneous non-intrathoracic placement relative to a patient's heart, and include detection circuitry and energy delivery circuitry. One or more electrodes configured for subcutaneous non-intrathoracic placement relative to the patient's heart may be coupled to the detection and energy delivery circuitry. A controller may be provided in the housing and coupled to the detection and energy delivery circuitry. The controller, in response to detection of a cardiac fibrillation event, coordinates delivery of a pre-shock waveform sufficient in energy to cause contraction of skeletal musculature in the patient's thorax but insufficient in energy to defibrillate the patient's heart. The system may then deliver a defibrillation waveform to the patient's heart during contraction of the skeletal musculature.

The electrodes may include a can electrode of the housing and at least one subcutaneous non-intrathoracic electrode coupled to the detection and energy delivery circuitry. In another embodiment, at least one subcutaneous non-intrathoracic electrode array is coupled to the detection and energy delivery circuitry via a lead. In a further embodiment, at least two subcutaneous non-intrathoracic electrodes are coupled to the detection and energy delivery circuitry. A capacitor module may store energy for the subcutaneous defibrillation therapy, with a total energy of the capacitor module dischargeable by the capacitor module divided equally or unequally between pre-shock waveform energy and defibrillation waveform energy.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac sensing and/or stimulation device as implanted in a patient in accordance with an embodiment of the present invention;

FIG. 1C is a block diagram illustrating various components of a transthoracic cardiac sensing and/or stimulation device in accordance with an embodiment of the present invention;

FIG. 1D is a block diagram illustrating various processing and detection components of a transthoracic cardiac sensing and/or stimulation device in accordance with an embodiment of the present invention;

FIG. 2 is a diagram illustrating components of a transthoracic cardiac sensing and/or stimulation device as implanted in a patient in accordance with an embodiment of the present invention;

FIG. 3A illustrates various waveforms associated with muscle contraction resulting from delivery of a shock;

FIG. 3B is a 1 second plot of the skeletal muscle motion signal and the defibrillation shock signal shown in FIG. 3A;

FIG. 3C is an expanded view of the skeletal muscle and shock waveforms shown in FIG. 3B;

FIG. 3D illustrates the application of a pre-shock waveform to initiate skeletal muscle contraction, followed by application of a defibrillation shock in accordance with an embodiment of the present invention;

FIG. 3E illustrates a method in accordance with the present invention employing a pre-shock waveform and defibrillation shock delivery; and

FIG. 3F illustrates an example of the pre-shock waveform and the defibrillation shock waveform being produced from the same defibrillation capacitor of a transthoracic cardiac sensing and/or stimulation device in accordance with an embodiment of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

An implanted device according to the present invention may include one or more of the features, structures, methods, or combinations thereof described hereinbelow. For example, a cardiac stimulator may be implemented to include one or more of the advantageous features and/or processes described below. It is intended that such a stimulator, or other implanted or partially implanted device need not include all of the features described herein, but may be implemented to include selected features that provide for unique structures and/or functionality. Such a device may be implemented to provide a variety of therapeutic or diagnostic functions.

In general terms, induced patient skeletal muscle contraction and cardiac defibrillation arrangements and methods in accordance with the present invention may be used with a subcutaneous cardiac monitoring and stimulation device. One such device is an implantable transthoracic cardiac sensing and/or stimulation (ITCS) device that may be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart.

The primary housing (e.g., the active or non-active can) of the ITCS device, for example, may be configured for positioning outside of the rib cage at an intercostal or subcostal location, within the abdomen, or in the upper chest region (e.g., subclavian location, such as above the third rib). In one implementation, one or more electrodes may be located on the primary housing and/or at other locations about, but not in direct contact with the heart, great vessel or coronary vasculature.

In another implementation, one or more leads incorporating electrodes may be located in direct contact with the heart, great vessel or coronary vasculature, such as via one or more leads implanted by use of conventional transvenous delivery approaches. In a further implementation, for example, one or more subcutaneous electrode subsystems or electrode arrays may be used to sense cardiac activity and deliver cardiac stimulation energy in an ITCS device configuration employing an active can or a configuration employing a non-active can. Electrodes may be situated at anterior and/or posterior locations relative to the heart.

Certain configurations illustrated herein are generally capable of implementing various functions traditionally performed by an ICD, and may operate in numerous cardioversion/defibrillation modes as are known in the art. Exemplary ICD circuitry, structures and functionality, aspects of which may be incorporated in an ITCS device of a type that may benefit from patient activity sensing in accordance with the present invention, are disclosed in commonly owned U.S. Pat. Nos. 5,133,353; 5,179,945; 5,314,459; 5,318,597; 5,620,466; and 5,662,688, which are hereby incorporated herein by reference in their respective entireties.

In particular configurations, systems and methods may perform functions traditionally performed by pacemakers, such as providing various pacing therapies as are known in the art, in addition to cardioversion/defibrillation therapies. Exemplary pacemaker circuitry, structures and functionality, aspects of which may be incorporated in an ITCS device of a type that may benefit from signal separation, are disclosed in commonly owned U.S. Pat. Nos. 4,562,841; 5,036,849; 5,284,136; 5,376,106; 5,540,727; 5,836,987; 6,044,298; and 6,055,454, which are hereby incorporated herein by reference in their respective entireties.

An ITCS device in accordance with the present invention may implement diagnostic and/or monitoring functions as well as provide cardiac stimulation therapy. Exemplary cardiac monitoring circuitry, structures and functionality, aspects of which may be incorporated in an ITCS device of a type that may benefit from information on patient activity in accordance with the present invention, are disclosed in commonly owned U.S. Pat. Nos. 5,313,953; 5,388,578; and 5,411,031, which are hereby incorporated herein by reference.

An ITCS device may be used to implement various diagnostic functions, which may involve performing rate-based, pattern and rate-based, and/or morphological tachyarrhythmia discrimination analyses. Subcutaneous, cutaneous, and/or external sensors may be employed to acquire physiologic and non-physiologic information for purposes of enhancing tachyarrhythmia detection and termination. It is understood that configurations, features, and combination of features described in the present disclosure may be implemented in a wide range of implantable medical devices, and that such embodiments and features are not limited to the particular devices described herein.

When comparing ICDs to external defibrillators, external defibrillation output requirements are greater than requirements for implantable ICDs that use intravenous electrodes extending into or on the heart. It is generally accepted that the heart is receiving only a percentage of the total defibrillation current when using external fibrillation versus using electrodes implanted in or on the heart. Subcutaneous electrode defibrillation may have requirements that are similar to external requirements. Methods for lowering the defibrillation requirements of a patient when using subcutaneous systems may be advantageous for practical implementation of such systems.

The respiratory ventilation cycle has been found to have an effect on transthoracic impedance and defibrillation threshold efficacy. In particular, it has been found that the expiratory portion of the respiration cycle reduces transthoracic impedance and defibrillation thresholds as compared with the inspiration portion of the respiration cycle.

As will be described below, sudden contraction of the skeletal muscles in the region of the thorax causes sudden respiratory expiration. Application of a defibrillation shock at the appropriate time during thoracic skeletal muscle contraction, such as at the time of maximum expiration, may lower the defibrillation threshold. Other beneficial effects from the skeletal muscle contraction may include shortening of skeletal muscle fibers and improved placement of the heart between subcutaneous electrodes further increasing defibrillation current density in the heart.

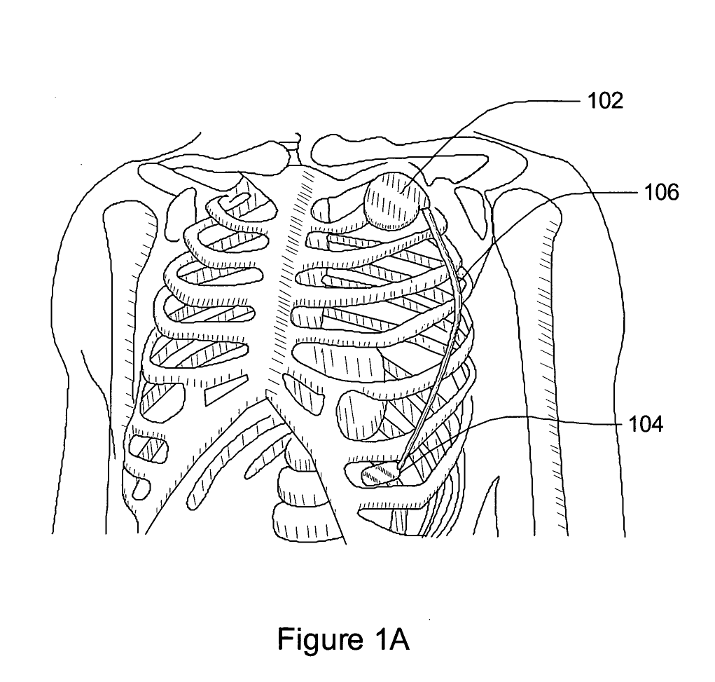

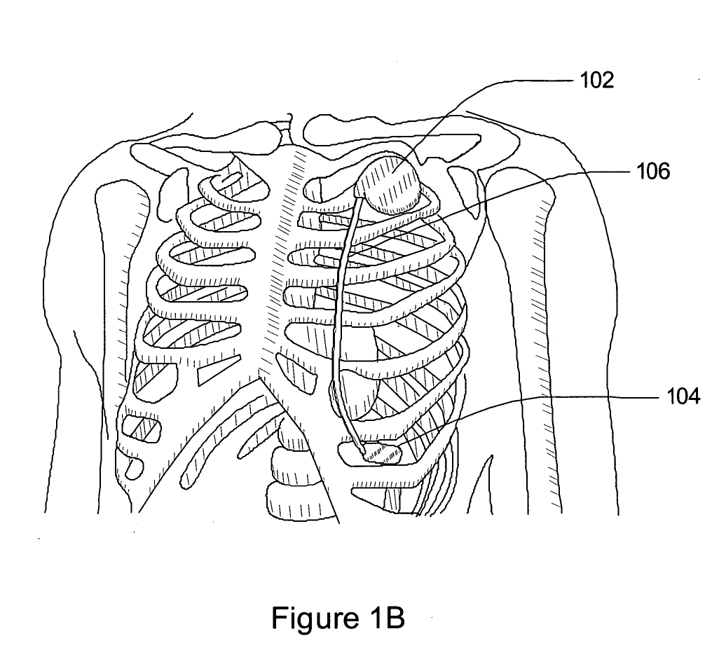

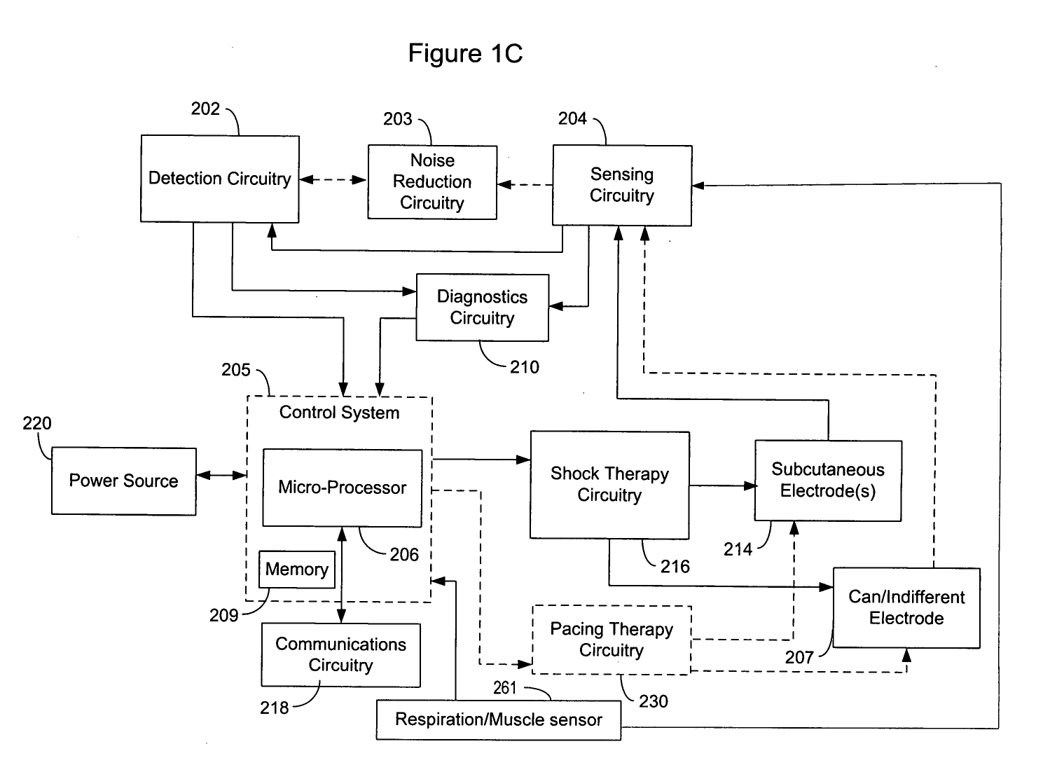

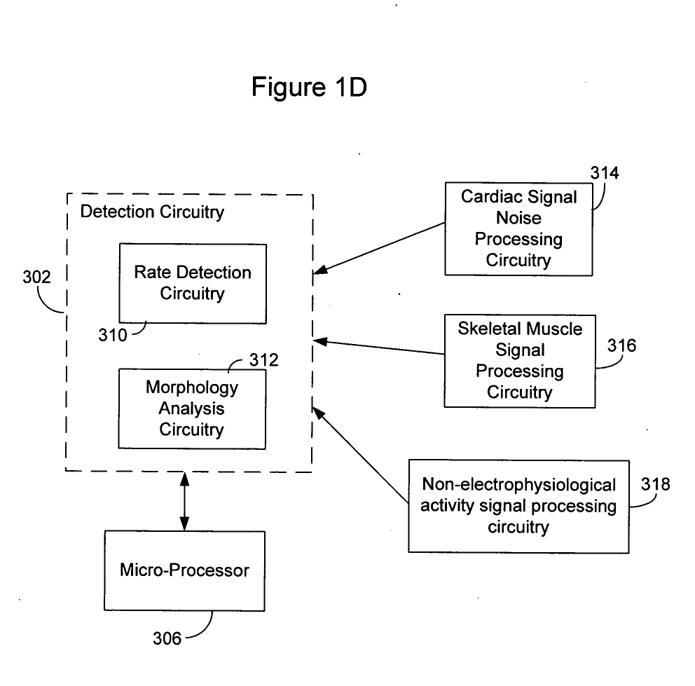

Referring now to FIGS. 1A and 1B of the drawings, there is shown a configuration of a subcutaneous ITCS device having components implanted in the chest region of a patient at different locations. In the particular configuration shown in FIGS. 1A and 1B, the ITCS device includes a housing 102 within which various cardiac sensing, detection, processing, and energy delivery circuitry may be housed. It is understood that the components and functionality depicted in the figures and described herein may be implemented in hardware, software, or a combination of hardware and software. It is further understood that the components and functionality depicted as separate or discrete blocks/elements in the figures may be implemented in combination with other components and functionality, and that the depiction of such components and functionality in individual or integral form is for purposes of clarity of explanation, and not of limitation.

Communications circuitry is disposed within the housing 102 for facilitating communication between the ITCS device and an external communication device, such as a portable or bed-side communication station, patient-carried/worn communication station, or external programmer, for example. The communications circuitry may also facilitate unidirectional or bidirectional communication with one or more external, cutaneous, or subcutaneous physiologic or non-physiologic sensors. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed.

In the configuration shown in FIGS. 1A and 1B, a subcutaneous electrode 104 may be positioned under the skin in the chest region and situated distal from the housing 102. The subcutaneous and, if applicable, housing electrode(s) may be positioned about the heart at various locations and orientations, such as at various anterior and/or posterior locations relative to the heart. The subcutaneous electrode 104 is coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes may be situated on the elongated structure of the electrode support, the housing 102, and/or the distal electrode assembly (shown as subcutaneous electrode 104 in the configuration shown in FIGS. 1A and 1B).

The electrode support assembly defines a physically separable unit relative to the housing 102. The electrode support assembly includes mechanical and electrical couplings that facilitate mating engagement with corresponding mechanical and electrical couplings of the housing 102. For example, a header block arrangement may be configured to include both electrical and mechanical couplings that provide for mechanical and electrical connections between the electrode support assembly and housing 102. The header block arrangement may be provided on the housing 102 or the electrode support assembly. Alternatively, a mechanical/electrical coupler may be used to establish mechanical and electrical connections between the electrode support assembly and housing 102. In such a configuration, a variety of different electrode support assemblies of varying shapes, sizes, and electrode configurations may be made available for physically and electrically connecting to a standard ITCS device housing 102.

It is noted that the electrodes and the lead assembly 106 may be configured to assume a variety of shapes. For example, the lead assembly 106 may have a wedge, chevron, flattened oval, or a ribbon shape, and the subcutaneous electrode 104 may include a number of spaced electrodes, such as an array or band of electrodes. Moreover, two or more subcutaneous electrodes 104 may be mounted to multiple electrode support assemblies 106 to achieve a desired spaced relationship amongst subcutaneous electrodes 104.

An ITCS device may incorporate circuitry, structures and functionality of the subcutaneous implantable medical devices disclosed in commonly owned U.S. Pat. Nos. 5,203,348; 5,230,337; 5,360,442; 5,366,496; 5,391,200; 5,397,342; 5,545,202; 5,603,732; and 5,916,243, which are hereby incorporated herein by reference in their respective entireties.

FIG. 1C is a block diagram depicting various components of an ITCS device in accordance with one configuration. According to this configuration, the ITCS device incorporates a processor-based control system 205 which includes a micro-processor 206 coupled to appropriate memory (volatile and/or non-volatile) 209, it being understood that any logic-based control architecture may be used. The control system 205 is coupled to circuitry and components to sense, detect, and analyze electrical signals produced by the heart and patient activity signals. The control system 205 is also configured to deliver electrical stimulation energy to the heart under predetermined conditions to treat cardiac arrhythmias. In certain configurations, the control system 205 and associated components also provide pacing therapy to the heart. The electrical energy delivered by the ITCS device may be in the form of low energy pacing pulses or high-energy pulses for cardioversion or defibrillation.

Electrocardiogram (ECG) signals and skeletal muscle signals are sensed using the subcutaneous electrode(s) 214 and/or the can or indifferent electrode 207 provided on the ITCS device housing. ECG and skeletal muscle signals may also be sensed using only the subcutaneous electrodes 214, such as in a non-active can configuration. As such, unipolar, bipolar, or combined unipolar/bipolar electrode configurations as well as multi-element electrodes and combinations of noise canceling and standard electrodes may be employed. The sensed ECG signals are received by sensing circuitry 204, which includes sense amplification circuitry and may also include filtering circuitry and an analog-to-digital (A/D) converter. The sensed ECG and skeletal muscle signals processed by the sensing circuitry 204 may be received by noise reduction circuitry 203, which may further reduce noise before signals are sent to the detection circuitry 202.

Noise reduction circuitry 203 may also be incorporated after detection circuitry 202 in cases where high power or computationally intensive noise reduction algorithms are required. The noise reduction circuitry 203, by way of amplifiers used to perform operations with the electrode signals, may also perform the function of the sensing circuitry 204. Combining the functions of sensing circuitry 204 and noise reduction circuitry 203 may be useful to minimize the necessary componentry and lower the power requirements of the system.

In the illustrative configuration shown in FIG. 1C, the detection circuitry 202 is coupled to, or otherwise incorporates, noise reduction circuitry 203. The noise reduction circuitry 203 operates to improve the signal-to-noise ratio of sensed signals by removing noise content of the sensed cardiac signals introduced from various sources.

Detection circuitry 202 typically includes a signal processor that coordinates analysis of the sensed cardiac signals and/or other sensor inputs to detect cardiac arrhythmias, such as, in particular, tachyarrhythmia. Rate based and/or morphological discrimination algorithms may be implemented by the signal processor of the detection circuitry 202 to detect and verify the presence and severity of an arrhythmic episode. Examples of arrhythmia detection and discrimination circuitry, structures, and techniques, aspects of which may be implemented by an ITCS device of a type that may benefit from patient activity sensing in accordance with the present invention, are disclosed in commonly owned U.S. Pat. Nos. 5,301,677 and 6,438,410, which are hereby incorporated herein by reference in their respective entireties.

The detection circuitry 202 communicates cardiac signal information to the control system 205. Memory circuitry 209 of the control system 205 contains parameters for operating in various sensing, defibrillation, and, if applicable, pacing modes, and stores data indicative of cardiac signals received by the detection circuitry 202. The memory circuitry 209 may also be configured to store historical ECG and therapy data, which may be used for various purposes and transmitted to an external receiving device as needed or desired.

In certain configurations, the ITCS device may include diagnostics circuitry 210. The diagnostics circuitry 210 typically receives input signals from the detection circuitry 202 and the sensing circuitry 204. The diagnostics circuitry 210 provides diagnostics data to the control system 205, it being understood that the control system 205 may incorporate all or part of the diagnostics circuitry 210 or its functionality. The control system 205 may store and use information provided by the diagnostics circuitry 210 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to therapy delivery.

According to a configuration that provides cardioversion and defibrillation therapies, the control system 205 processes cardiac signal data received from the detection circuitry 202 and initiates appropriate tachyarrhythmia therapies to terminate cardiac arrhythmic episodes and return the heart to normal sinus rhythm. The control system 205 is coupled to shock therapy circuitry 216. The shock therapy circuitry 216 is coupled to the subcutaneous electrode(s) 214 and the can or indifferent electrode 207 of the ITCS device housing. Upon command, the shock therapy circuitry 216 delivers cardioversion and defibrillation stimulation energy to the heart in accordance with a selected cardioversion or defibrillation therapy. In a less sophisticated configuration, the shock therapy circuitry 216 is controlled to deliver defibrillation therapies, in contrast to a configuration that provides for delivery of both cardioversion and defibrillation therapies. Examples of ICD high energy delivery circuitry, structures and functionality, aspects of which may be incorporated in an ITCS device of a type that may benefit from aspects of the present invention are disclosed in commonly owned U.S. Pat. Nos. 5,372,606; 5,411,525; 5,468,254; and 5,634,938, which are hereby incorporated herein by reference.

In accordance with another configuration, an ITCS device may incorporate a cardiac pacing capability in addition to cardioversion and/or defibrillation capabilities. As is shown in dotted lines in FIG. 1C, the ITCS device may include pacing therapy circuitry 230, which is coupled to the control system 205 and the subcutaneous and can/indifferent electrodes 214, 207. Upon command, the pacing therapy circuitry delivers pacing pulses to the heart in accordance with a selected pacing therapy. Control signals, developed in accordance with a pacing regimen by pacemaker circuitry within the control system 205, are initiated and transmitted to the pacing therapy circuitry 230 where pacing pulses are generated.

A number of cardiac pacing therapies may be useful in a transthoracic cardiac monitoring and/or stimulation device. Such cardiac pacing therapies may be delivered via the pacing therapy circuitry 230 as shown in FIG. 1C. Alternatively, cardiac pacing therapies may be delivered via the shock therapy circuitry 216, which effectively obviates the need for separate pacemaker circuitry.

The ITCS device shown in FIG. 1C is configured to receive signals from one or more physiologic and/or non-physiologic sensors 261 used to sense, for example, skeletal muscle movement, transthoracic impedance, or other parameters indicative of patient respiration in accordance with embodiments of the present invention. Depending on the type of sensor employed, signals generated by the sensors may be communicated to transducer circuitry coupled directly to the detection circuitry 202 or indirectly via the sensing circuitry 204. It is noted that certain sensors may transmit sense data to the control system 205 without processing by the detection circuitry 202.

Sensors for detecting patient respiration, skeletal muscle contraction, transthoracic impedance, or other sensors useful for coordinated pre-shock and shock waveform delivery may be coupled directly to the detection circuitry 202 or indirectly via the sensing circuitry 204. One or more respiration/muscle sensors may sense patient activity, such as respiration, movement, or other parameters. Examples of useful non-cardiac sensors are skeletal muscle specific electrodes, electromyogram sensors, acoustic sensors and/or pressure transducers, accelerometers, and transthoracic impendence sensing arrangements. Signals from these sensors may be used to detect patient respiration cycle, movement, position, or the like. A respiration/muscle sensor 261 is illustrated in FIG. 1C connected to one or both of the sensing circuitry 204 and the control system 205.

Communications circuitry 218 is coupled to the microprocessor 206 of the control system 205. The communications circuitry 218 allows the ITCS device to communicate with one or more receiving devices or systems situated external to the ITCS device. By way of example, the ITCS device may communicate with a patient-worn, portable or bedside communication system via the communications circuitry 218. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the ITCS device via the communications circuitry 218. It is noted that physiologic or non-physiologic sensors equipped with wireless transmitters or transceivers may communicate with a receiving system external of the patient.

The communications circuitry 218 may allow the ITCS device to communicate with an external programmer. In one configuration, the communications circuitry 218 and the programmer unit (not shown) use a wire loop antenna and a radio frequency telemetric link, as is known in the art, to receive and transmit signals and data between the programmer unit and communications circuitry 218. In this manner, programming commands and data are transferred between the ITCS device and the programmer unit during and after implant. Using a programmer, a physician is able to set or modify various parameters used by the ITCS device. For example, a physician may set or modify parameters affecting sensing, detection, pacing, and defibrillation functions of the ITCS device, including pacing and cardioversion/defibrillation therapy modes.

Typically, the ITCS device is encased and hermetically sealed in a housing suitable for implanting in a human body as is known in the art. Power to the ITCS device is supplied by an electrochemical power source 220 housed within the ITCS device. In one configuration, the power source 220 includes a rechargeable battery. According to this configuration, charging circuitry is coupled to the power source 220 to facilitate repeated non-invasive charging of the power source 220. The communications circuitry 218, or separate receiver circuitry, is configured to receive radio-frequency (RF) energy transmitted by an external RF energy transmitter. The ITCS device may, in addition to a rechargeable power source, include a non-rechargeable battery. It is understood that a rechargeable power source need not be used, in which case a long-life non-rechargeable battery is employed.

FIG. 1D illustrates a configuration of detection circuitry 302 of an ITCS device, which includes one or both of rate detection circuitry 310 and morphological analysis circuitry 312. Detection and verification of arrhythmias may be accomplished using rate-based discrimination algorithms as known in the art implemented by the rate detection circuitry 310. Arrhythmic episodes may also be detected and verified by morphology-based analysis of sensed cardiac signals as is known in the art. Tiered or parallel arrhythmia discrimination algorithms may also be implemented using both rate-based and morphologic-based approaches. Further, a rate and pattern-based arrhythmia detection and discrimination approach may be employed to detect and/or verify arrhythmic episodes, such as the approach disclosed in U.S. Pat. Nos. 6,487,443; 6,259,947; 6,141,581; 5,855,593; and 5,545,186, which are hereby incorporated herein by reference in their respective entireties.

The detection circuitry 302, which is coupled to a microprocessor 306, may be configured to incorporate, or communicate with, specialized circuitry for processing sensed signals in manners particularly useful in a transthoracic cardiac sensing and/or stimulation device. As is shown by way of example in FIG. 1D, the detection circuitry 302 may receive information from multiple physiologic and non-physiologic sensors. Non-electrophysiological signals, such as from accelerometers, position sensors, movement sensors, or other patient activity monitoring sensors, may be detected and processed by non-electrophysiological activity signal processing circuitry 318 for a variety of purposes. The signals are transmitted to the detection circuitry 302, via a hardwire or wireless link, and used to coordinate defibrillation therapy with the patient's natural or induced respiration cycle in accordance with the present invention.

The detection circuitry 302 may also receive patient activity information from one or more sensors that monitor patient activity, such as electromyogram signals. In addition to ECG signals, transthoracic electrodes readily detect skeletal muscle signals. Such skeletal muscle signals may be used in accordance with the present invention to determine the breathing cycle of the patient. Processing circuitry 316 receives signals from one or more patient activity sensors, and transmits processed patient activity signal data to the detection circuitry 302.

In accordance with embodiments of the invention, an ITCS device may be implemented to include a subcutaneous electrode system that provides for one or both of cardiac sensing and arrhythmia therapy delivery in combination with patient activity sensing, such as skeletal muscle signal sensing or transthoracic impedance signal sensing. According to one approach, an ITCS device may be implemented as a chronically implantable system that performs monitoring, diagnostic and/or therapeutic functions. The ITCS device may automatically detect and treat cardiac arrhythmias. In one configuration, the ITCS device includes a pulse generator and one or more electrodes that are implanted subcutaneously in the chest region of the body, such as in the anterior thoracic region of the body. The ITCS device may be used to provide atrial and ventricular therapy for bradycardia and/or tachycardia arrhythmias. Tachyarrhythmia therapy may include cardioversion, defibrillation and anti-tachycardia pacing (ATP), for example, to treat atrial or ventricular tachycardia or fibrillation. Bradycardia therapy may include temporary post-shock pacing for bradycardia or asystole. Methods and systems for implementing post-shock pacing for bradycardia or asystole are described in commonly owned U.S. Patent Application entitled “Subcutaneous Cardiac Stimulator Employing Post-Shock Transthoracic Asystole Prevention Pacing, Ser. No. 10/377,274, filed on Feb. 28, 2003, which is incorporated herein by reference in its entirety.

In one configuration, an ITCS device according to one approach may utilize conventional pulse generator and subcutaneous electrode implant techniques. The pulse generator device and electrodes may be chronically implanted subcutaneously. Such an ITCS device may be used to automatically detect and treat arrhythmias similarly to conventional implantable systems. In another configuration, the ITCS device may include a unitary structure (e.g., a single housing/unit). The electronic components and electrode conductors/connectors are disposed within or on the unitary ITCS device housing/electrode support assembly.

The ITCS device contains the electronics and may be similar to a conventional implantable defibrillator. High voltage shock therapy may be delivered between two or more electrodes, one of which may be the pulse generator housing (e.g., can), placed subcutaneously in the thoracic region of the body.

Additionally or alternatively, the ITCS device may also provide lower energy electrical stimulation for bradycardia therapy. The ITCS device may provide brady pacing similarly to a conventional pacemaker. The ITCS device may provide temporary post-shock pacing for bradycardia or asystole. Sensing and/or pacing may be accomplished using sense/pace electrodes positioned on an electrode subsystem also incorporating shock electrodes, or by separate electrodes implanted subcutaneously.

The ITCS device may detect a variety of physiological signals that may be used in connection with various diagnostic, therapeutic or monitoring implementations. For example, the ITCS device may include sensors or circuitry for detecting respiratory system signals, cardiac system signals, and signals related to patient activity. In one embodiment, the ITCS device senses intrathoracic impedance, from which various respiratory parameters may be derived, including, for example, respiratory tidal volume and minute ventilation. Sensors and associated circuitry may be incorporated in connection with an ITCS device for detecting one or more body movement or body position related signals. For example, accelerometers and GPS devices may be employed to detect patient activity, patient location, body orientation, or torso position.

The ITCS device may be used within the structure of an advanced patient management (APM) system. Advanced patient management systems may allow physicians to remotely and automatically monitor cardiac and respiratory functions, as well as other patient conditions. In one example, implantable cardiac rhythm management systems, such as cardiac pacemakers, defibrillators, and resynchronization devices, may be equipped with various telecommunications and information technologies that enable real-time data collection, diagnosis, and treatment of the patient. Various embodiments described herein may be used in connection with advanced patient management. Methods, structures, and/or techniques described herein, which may be adapted to provide for remote patient/device monitoring, diagnosis, therapy, or other APM related methodologies, may incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

An ITCS device according to one approach provides an easy to implant therapeutic, diagnostic or monitoring system. The ITCS system may be implanted without the need for intravenous or intrathoracic access, providing a simpler, less invasive implant procedure and minimizing lead and surgical complications. In addition, this system would have advantages for use in patients for whom transvenous lead systems cause complications. Such complications include, but are not limited to, surgical complications, infection, insufficient vessel patency, complications associated with the presence of artificial valves, and limitations in pediatric patients due to patient growth, among others. An ITCS system according to this approach is distinct from conventional approaches in that it may be configured to include a combination of two or more electrode subsystems that are implanted subcutaneously in the anterior thorax.

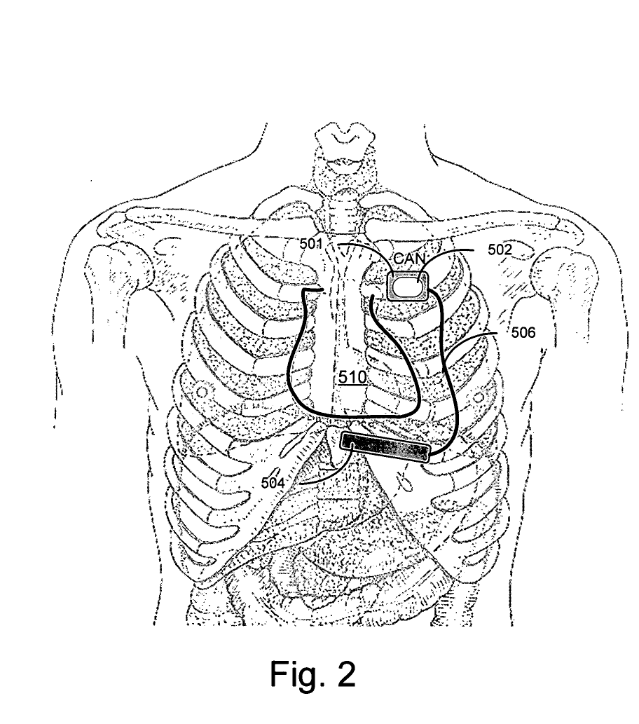

In one configuration, as is illustrated in FIG. 2, electrode subsystems of an ITCS system are arranged about a patient's heart 510. The ITCS system includes a first electrode subsystem 502, including a can electrode configured for the sensing of skeletal muscle activity, transthoracic impedance, or the like, and a second electrode subsystem 504 that includes one or more electrodes. The electrode subsystems 502, 504 may include a number of electrodes used for sensing and/or electrical stimulation.

In various configurations, the second electrode subsystem 504 may include a combination of electrodes. The combination of electrodes of the second electrode subsystem 504 may include coil electrodes, tip electrodes, ring electrodes, multi-element coils, spiral coils, spiral coils mounted on non-conductive backing, screen patch electrodes, and other electrode configurations. A suitable non-conductive backing material is silicone rubber, for example.

The first electrode subsystem 502 is positioned on the housing 501 that encloses the ITCS device electronics. In one embodiment, the first electrode subsystem 502 includes the entirety of the external surface of housing 501. In other embodiments, various portions of the housing 501 may be electrically isolated from the first electrode subsystem 502 or from tissue. For example, the active area of the first electrode subsystem 502 may include all or a portion of either the anterior or posterior surface of the housing 501 to direct current flow in a manner advantageous for sensing cardiac activity, sensing skeletal muscle activity, and/or providing coordinated skeletal muscle contraction and cardiac stimulation therapy.

In accordance with one embodiment, the housing 501 may resemble that of a conventional implantable ICD, is approximately 20-100 cc in volume, with a thickness of 0.4 to 2 cm and with a surface area on each face of approximately 30 to 100 cm². As previously discussed, portions of the housing may be electrically isolated from tissue to optimally direct current flow and/or provide shielding for specific directivity. For example, portions of the housing 501 may be covered with a non-conductive, or otherwise electrically resistive, material to direct current flow. Suitable non-conductive material coatings include those formed from silicone rubber, polyurethane, or parylene, for example.

In an ITCS device configured in accordance with an embodiment of the present invention, subcutaneous defibrillation timing may be correlated with induced skeletal muscle contraction to provide several advantages over other approaches. An ITCS device configured to provide subcutaneous defibrillation may be implemented to stimulate skeletal musculature so that contraction occurs before and/or during defibrillation shock delivery.

Prior to delivery of a subcutaneous defibrillation shock, an ITCS device may deliver a short electrical pulse across the defibrillation electrodes, which, after a delay of less then 100 milliseconds, for example, causes contraction of the skeletal muscles. A defibrillation shock is applied when the skeletal muscular system is contracted. This contraction should cause muscle fiber shortening, lung deflation, and placement of the heart in a more optimal position for receiving increased shock current.

Timing a defibrillation shock in this manner has the potential to decrease defibrillation thresholds. If defibrillation thresholds can be reduced in a subcutaneous defibrillation system using this technique, device energy requirements may be lowered and patients may receive defibrillation shocks with increased effectiveness.

Some studies have shown that a significantly higher transthoracic impedance results during respiratory inspiration, and a significant decrease in defibrillation success rate results when shocks are delivered during inspiration when compared with expiration. A subcutaneous defibrillation system may advantageously provide for reduced defibrillation thresholds by providing a pre-shock waveform as a method of stimulating skeletal musculature so that the defibrillation shock occurs during or after muscle contraction.

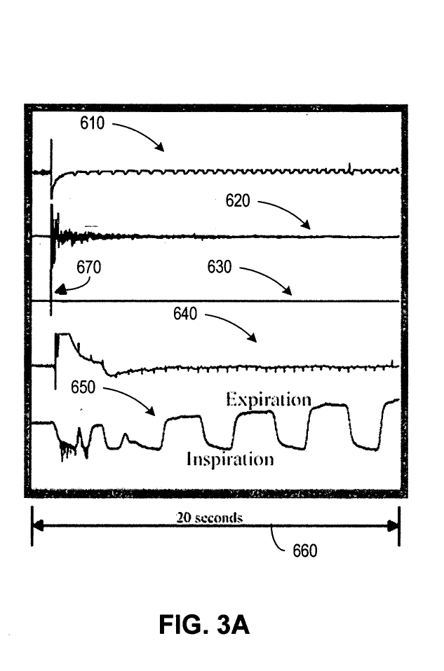

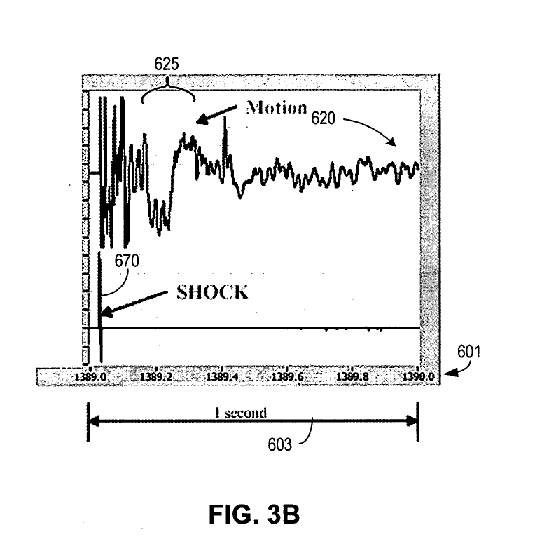

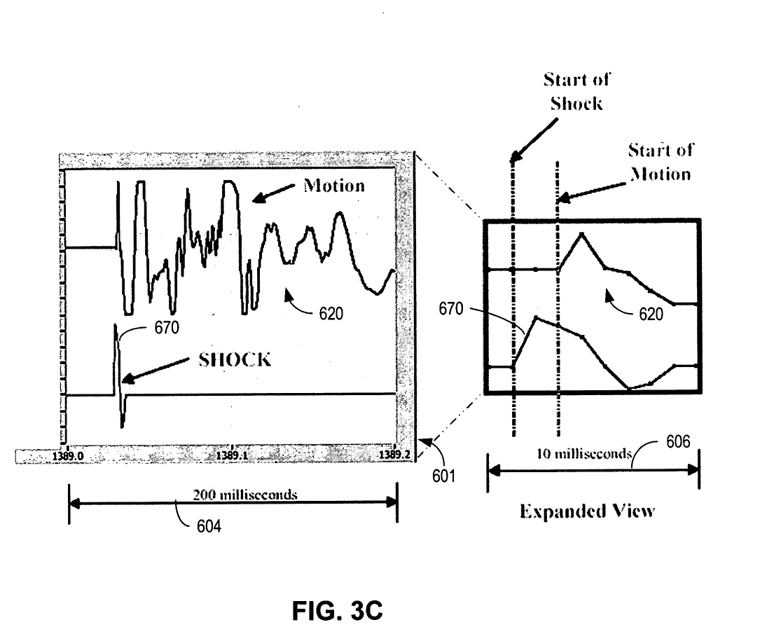

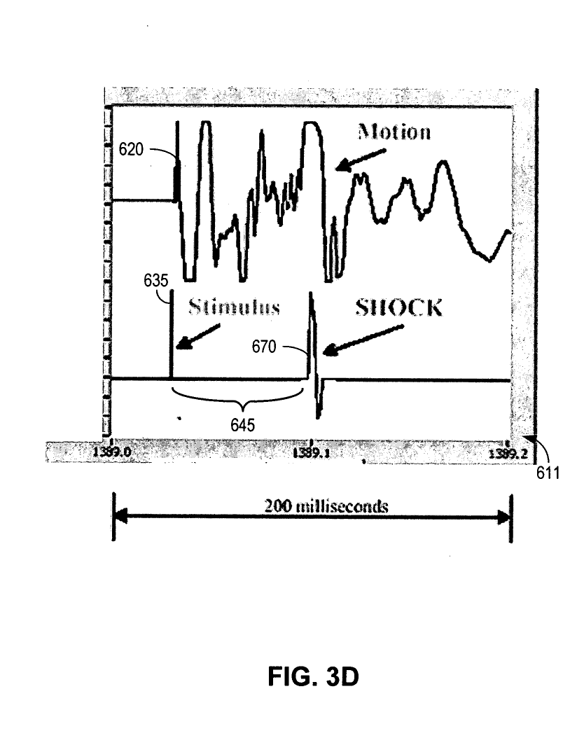

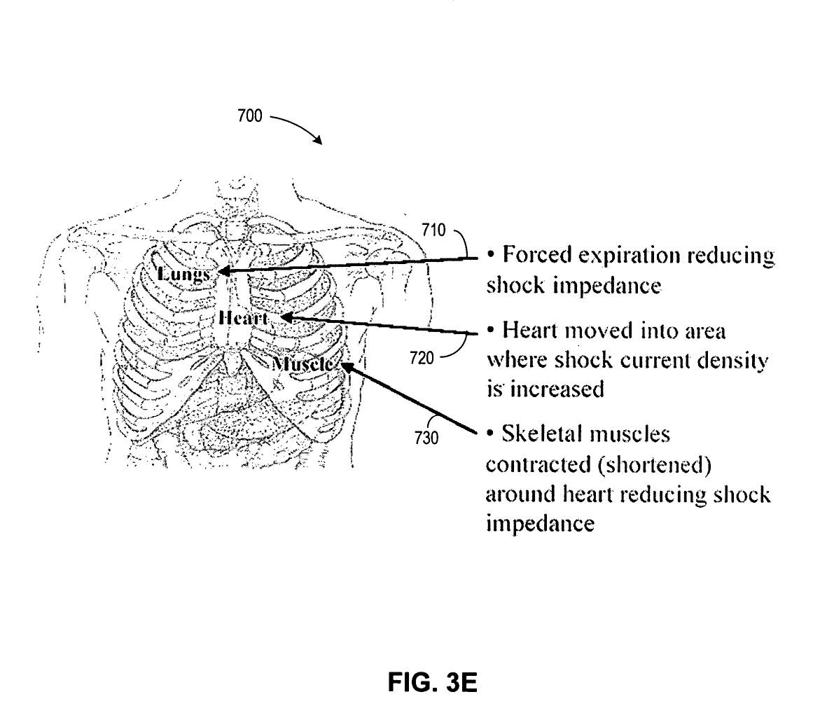

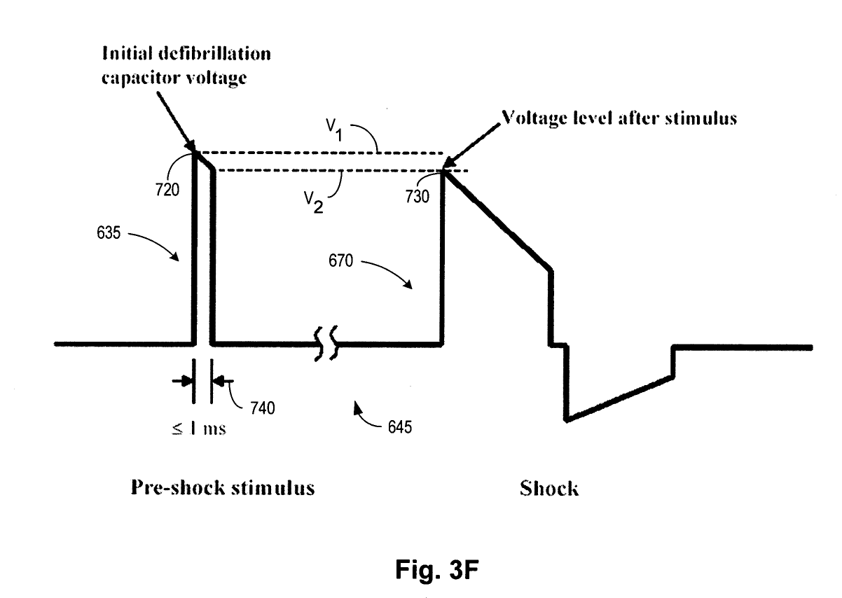

FIGS. 3A-3F illustrate various aspects of an ITCS device that is implemented to deliver an electrical pulse to precondition skeletal musculature via induced contraction preceding delivery of a subcutaneous defibrillation shock in accordance with the present invention. FIG. 3A shows various waveforms associated with muscle contraction resulting from delivery of a shock. The signal information shown in FIG. 3A includes a ventricular signal 610, a skeletal muscle motion signal 620 (via an accelerometer type sensor), a defibrillation voltage 630 (e.g., shock waveform), a surface EKG signal 640, and a respirator-induced ETCO₂ signal 650, which is an indirect measure of inspiration and expiration. These signals are plotted over a 20 second time period 660 following delivery of a defibrillation shock 670.

FIG. 3B is a 1 second time period 603 plot of the skeletal muscle motion signal 620 and the defibrillation shock signal 670 shown in FIG. 3A. The abscissa 601 of FIG. 3B is time, given in units of seconds, for both the skeletal muscle motion signal 620 and the defibrillation shock 670 signal. FIG. 3B demonstrates that the greatest skeletal muscle contraction occurs within the first 200 milliseconds after the defibrillation shock 670 in this case, as indicated by the relatively large amplitude peaks of the skeletal muscle motion signal 620 during a time frame 625.

FIG. 3C illustrates a 200 millisecond time period 604 and a 10 millisecond time period 606 of the skeletal muscle motion signal 620 and the defibrillation shock 670 signal shown in FIG. 3B. The abscissa 601 of FIG. 3C is time, given in units of seconds, for both the skeletal muscle motion signal 620 and the defibrillation shock 670 signal, and taken from within the time frame of FIG. 3B. FIG. 3C shows that the skeletal muscle motion contraction starts after the peak of the defibrillation shock 670 in this case. As is shown in the expanded view of the 10 millisecond time period 606, the skeletal muscle motion signal 620 (upper signal) indicates that skeletal muscle motion (i.e., contraction) starts about 2 milliseconds after the start of the defibrillation shock 670.

FIG. 3D illustrates the application of a pre-shock waveform 635 to initiate skeletal muscle contraction, followed by application of the defibrillation shock 670. The abscissa 611 of FIG. 3D is time, given in units of seconds, for both the skeletal muscle motion signal 620 and the defibrillation shock signal 670.

A time delay 645 between the pre-shock waveform 635 and the defibrillation shock 670 delivery may be a constant time delay or may be selected so that delivery of the defibrillation shock 670 occurs during a particular portion of the contraction period of the skeletal muscle motion. Advantageous timing, such as the delay 645, may be defined by significant peak amplitude variations in the motion signal or other parameters such as a given change in measured impedance across the thorax. In the illustrative example shown in FIG. 3D, the defibrillation shock 670 is delivered after the delay 645 (in this example, about 70 milliseconds) from delivery of the pre-shock waveform 635. It is understood that the time delay 645 between the pre-shock waveform 635 and the defibrillation shock 670 delivery may vary from patient to patient and/or shock to shock.

FIG. 3E describes a method 700 associated with employment of the above-described pre-shock waveform and defibrillation shock delivery timing method. For example, application of the pre-shock waveform 635 may cause a forced expiration 710, which reduces the defibrillation shock impedance. The heart may be physically moved 720 into an area where the shock current density is greater. Moreover, skeletal muscle motion contraction could result in shorter conduction pathways around the heart, thereby reducing defibrillation shock impendence and/or providing an increased current density 730 in the heart.

FIG. 3F illustrates an example of the pre-shock waveform 635 and the defibrillation shock 670 waveform being produced from the same defibrillation capacitor of an ITCS device in accordance with an embodiment of the present invention. According to this approach, the capacitor is charged to an initial voltage 720, designated V₁, in response to the ITCS device detecting an arrhythmic condition warranting cardioversion or defibrillation therapy. After confirming the presence of such an arrhythmic condition, the ITCS device delivers the pre-shock waveform 635, for example, having a duration 740 of less than or equal to about 1 millisecond.

The pre-shock waveform 635 reduces the capacitor voltage from the initial voltage 720, designated V₁, to a voltage 730, designated V₂. After an appropriate delay 645, such as about 70 milliseconds, the defibrillation shock 670 is delivered from the capacitor beginning at the voltage 730 (V₂). The defibrillation shock 670 may be monophasic, biphasic (as shown in FIG. 3F) or other multiphasic form (e.g., triphasic), and have a desired tilt. In one approach, for example, a triphasic shock may be developed by combining the pre-shock waveform 635 and the main defibrillation shock 670. In another approach, the amplitude of the pre-shock waveform 635 may be selected independently of the defibrillation shock 670 voltage so that it affects a desired degree of skeletal muscle contraction, with minimal interaction with the cardiac conduction system.

The pre-shock waveform 635 may be delivered by a first vector, particularly suited for inducing skeletal muscle contraction, and the defibrillation shock 670 may be delivered by the same vector, or by a vector particularly suited for defibrillation of the patient's heart. Methods and devices for determining suitable vectors for skeletal muscle signal detection/stimulation and cardiac signal detection/stimulation are further described in commonly owned, co-pending U.S. patent application Ser. No. 10/738,608, filed Dec. 17, 2003 [Attorney Docket GUID.603PA]; and Ser. No. 10/799,341, filed Mar. 12, 2004 [Attorney Docket GUID.627PA]; and in U.S. Patent Application entitled “Subcutaneous Cardiac Stimulation System with Patient Activity Sensing,” filed Apr. 1, 2004 under Attorney Docket GUID.610 PA, which are hereby incorporated herein by reference.

A typical skeletal muscle action potential lasts about 1 to 2 milliseconds, with a total latent period lasting about 10 milliseconds before there is mechanical activity. This mechanical activity, as a function of muscle fiber shortening, typically starts at about 10 milliseconds and ends at about 100 milliseconds after the onset of the action potential. A peak shortening of skeletal muscle typically occurs at about 80 milliseconds.

The strength-duration of pre-shock waveform 635 that produces a skeletal muscle action potential may typically be in the range of about 70 to 400 microseconds, and may include durations up to 2 milliseconds or more. A typical pre-shock waveform 635 may have a minimum threshold current at a pulse-width that is greater than about 400 microseconds to more than 2 milliseconds for a square wave. The amplitude of the pre-shock waveform 635 at a given pulse width does not produce an action potential until the excitable cell membrane threshold is reached, with no further effect on that action potential at greater amplitudes. However, an increase in voltage invokes a larger number of muscle cells away from the electrodes which affects the overall pre-shock contraction.

The pre-shock waveform 635 energy used for skeletal muscle contraction typically represents less than about 10% of the total energy. For example, pre-shock waveforms having a pulse width of less than 0.3 millisecond as part of the defibrillation waveform, or pre-shock waveforms that are considerably less in amplitude than what is delivered for defibrillation, typically use less than 10% of the total energy available for a defibrillation shock therapy in an ITCS device.

It may be useful to provide more than 10% of the energy in a pre-shock waveform useful for skeletal muscle contraction. For example, the pre-shock waveform may have a pulse width equal to or less than about 2 milliseconds when the pre-shock waveform is derived from the first portion of the defibrillation shock waveform. In this example, 2 milliseconds may represent about 42% of the total energy (e.g., 2 milliseconds out of a total of 10 milliseconds) if the amplitude of the pre-shock waveform is that of what is stored on the defibrillation capacitor. If the pre-shock waveform is derived from the first portion of the defibrillation shock waveform, then it may be desirable to have a much shorter pulse width, such as less than about 1 millisecond, which may consume about 25% of the total energy available.

From the discussion above, it is understood that the pre-shock waveform 635 may have an initial amplitude greater than that of the defibrillation waveform 670. It is also understood that the pre-shock waveform 635 may have an initial amplitude less than that of the defibrillation waveform 670 or equal to that of the defibrillation waveform 670. In an embodiment in which the defibrillation waveform 670 has a first phase and a second phase, an energy level of one or both of the first phase and the second phase of the defibrillation waveform 670 may be reduced by an amount of energy corresponding to the energy level of the pre-shock waveform 635. In this regard, the loss of energy corresponding to the pre-shock waveform energy may be taken from the first phase and/or the second phase of the total defibrillation waveform energy.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A method of delivering a subcutaneous defibrillation therapy, comprising:
delivering, from a subcutaneous non-intrathoracic location relative to a patient's heart, a pre-shock waveform sufficient in energy such that contraction of skeletal musculature occurs in the patient's thorax, but insufficient in energy for defibrillating the patient's heart; and
delivering, from the subcutaneous non-intrathoracic location or other subcutaneous non-intrathoracic location, a defibrillation waveform to the patient's heart during contraction of the skeletal musculature.

2. The method of claim 1, wherein the pre-shock waveform is sufficient in energy such that deflation of the patient's lungs occurs.

3. The method of claim 1, wherein the pre-shock waveform is sufficient in energy such that muscle fiber shortening of the skeletal musculature occurs.

4. The method of claim 1, further comprising providing electrodes configured for subcutaneous non-intrathoracic placement in the patient, wherein the pre-shock waveform is sufficient in energy such that displacement of the patient's heart relative to the electrodes occurs.

5. The method of claim 4, wherein displacement of the patient's heart relative to the electrodes achieves an increased defibrillation current density in the patient's heart relative to a defibrillation current density in the patient's heart achievable in the absence of displacement of the patient's heart.

6. The method of claim 1, wherein the pre-shock waveform is sufficient in energy such that contraction of the skeletal musculature occurs, and reduces a defibrillation threshold relative to a defibrillation threshold associated with delivery of the defibrillation waveform in the absence of the skeletal musculature contraction.

7. The method of claim 1, further comprising initiating a delay interval relative to delivery of the pre-shock waveform, wherein the defibrillation waveform is delivered following expiration of the delay interval.

8. The method of claim 7, wherein the delay interval has a pre-established duration.

9. The method of claim 7, wherein the delay interval has a duration equal to or less than about 2 seconds.

10. The method of claim 7, wherein the delay interval has a duration equal to or less than about 200 milliseconds.

11. The method of claim 7, wherein the delay interval has a duration equal to or less than about 100 milliseconds.

12. The method of claim 1, wherein the pre-shock waveform has a pulse width equal to or less than about 2 milliseconds.

13. The method of claim 1, wherein the pre-shock waveform has a pulse width equal to or less than about 20% of that of the defibrillation waveform.

14. The method of claim 1, wherein the pre-shock waveform has an initial amplitude greater than that of the defibrillation waveform.

15. The method of claim 1, wherein the pre-shock waveform has an initial amplitude less than that of the defibrillation waveform.

16. The method of claim 1, wherein the defibrillation waveform comprises a multiphasic defibrillation waveform.

17. The method of claim 1, wherein the defibrillation waveform comprises a biphasic, truncated exponential defibrillation waveform.

18. The method of claim 1, wherein the pre-shock waveform has an energy level, and the defibrillation waveform has a first phase and a second phase, an energy level of one or both of the first phase and the second phase of the defibrillation waveform reduced by an amount of energy corresponding to the energy level of the pre-shock waveform.

19. The method of claim 1, wherein the pre-shock waveform is delivered using a first vector, and the defibrillation waveform is delivered using the first vector.

20. The method of claim 1, wherein the pre-shock waveform is delivered using a first vector, and the defibrillation waveform is delivered using a second vector differing from the first vector.

21. The method of claim 1, further comprising detecting motion of the patient's thorax responsive to the pre-shock waveform, wherein delivering the defibrillation waveform is coordinated in relation to the detected motion.

22. The method of claim 21, further comprising coordinating delivery of the defibrillation waveform in relation to detection of a peak in the thoracic motion.

23. The method of claim 22, wherein coordinating delivery of the defibrillation waveform in relation to detection of the peak in the thoracic motion comprises using a pre-determined delay interval between delivery of the defibrillation and pre-shock waveforms.

24. The method of claim 1, further comprising:
detecting expiration of the patient's lungs responsive to the pre-shock waveform using transthoracic impedance; and
coordinating delivery of the defibrillation waveform in response to the detected expiration.

25. The method of claim 24, further comprising coordinating delivery of the defibrillation waveform in relation to detection of a minimum in the transthoracic impedance.

26. A system for delivering a subcutaneous defibrillation therapy, comprising:
a housing configured for subcutaneous non-intrathoracic placement relative to a patient's heart;
detection circuitry provided in the housing;
energy delivery circuitry provided in the housing;
one or more electrodes configured for subcutaneous non-intrathoracic placement relative to the patient's heart, the one or more electrodes coupled to the detection and energy delivery circuitry; and
a controller provided in the housing and coupled to the detection and energy delivery circuitry, the controller, in response to detection of a cardiac fibrillation event, coordinating delivery of a pre-shock waveform sufficient in energy such that contraction of skeletal musculature occurs in the patient's thorax but insufficient in energy for defibrillating the patient's heart, and delivery of a defibrillation waveform to the patient's heart during contraction of the skeletal musculature.

27. The system of claim 26, wherein the one or more electrodes comprise a can electrode of the housing and at least one subcutaneous non-intrathoracic electrode coupled to the detection and energy delivery circuitry.

28. The system of claim 26, wherein the one or more electrodes comprise a can electrode of the housing and at least one subcutaneous non-intrathoracic electrode array coupled to the detection and energy delivery circuitry via a lead.

29. The system of claim 26, wherein the one or more electrodes comprise at least two subcutaneous non-intrathoracic electrodes coupled to the detection and energy delivery circuitry.

30. The system of claim 26, wherein the energy delivery circuitry comprises a capacitor module that stores energy for the subcutaneous defibrillation therapy, a total energy of the capacitor module dischargeable by the capacitor module divided between pre-shock waveform energy and defibrillation waveform energy.

31. The system of claim 30, wherein the pre-shock waveform energy represents less than about 10% of the total energy.

32. The system of claim 26, wherein the pre-shock waveform has a pulse width equal to or less than about 2 milliseconds.

33. The system of claim 26, wherein the pre-shock waveform has a pulse width equal to or less than about 20% of that of the defibrillation waveform.

34. The system of claim 26, wherein the pre-shock waveform has an initial amplitude greater than that of the defibrillation waveform.

35. The system of claim 26, wherein the pre-shock waveform has an initial amplitude less than that of the defibrillation waveform.

36. The system of claim 26, wherein the defibrillation waveform comprises a multiphasic defibrillation waveform.

37. The system of claim 26, wherein the defibrillation waveform comprises a biphasic, truncated exponential defibrillation waveform.

38. The system of claim 26, wherein the pre-shock waveform has an energy level, and the defibrillation waveform has a first phase and a second phase, an energy level of one or both of the first phase and the second phase of the defibrillation waveform reduced by an amount of energy corresponding to the energy level of the pre-shock waveform.

39. The system of claim 26, wherein the pre-shock waveform is sufficient in energy such that deflation of the patient's lungs occurs.

40. The system of claim 26, wherein the pre-shock waveform is sufficient in energy such that muscle fiber shortening of the skeletal musculature occurs.

41. The system of claim 26, wherein the pre-shock waveform is sufficient in energy such that displacement of the patient's heart relative to the one or more electrodes occurs.

42. The system of claim 41, wherein the patient's heart is displaced relative to the one or more electrodes to achieve an increased defibrillation current density in the patient's heart relative to a defibrillation current density in the patient's heart achievable in the absence of displacement of the patient's heart.

43. The system of claim 26, wherein the pre-shock waveform is sufficient in energy such that contraction of the skeletal musculature occurs and reduces a defibrillation threshold relative to a defibrillation threshold associated with delivery of the defibrillation waveform in the absence of the skeletal musculature contraction.

44. The system of claim 26, wherein the controller initiates a delay interval relative to delivery of the pre-shock waveform, wherein the defibrillation waveform is delivered following expiration of the delay interval.

45. The system of claim 44, wherein the delay interval has a pre-established duration.

46. The system of claim 44, wherein the delay interval has a duration equal to or less than about 2 seconds.

47. The system of claim 44, wherein the delay interval has a duration equal to or less than about 200 milliseconds.

48. The system of claim 44, wherein the delay interval has a duration equal to or less than about 100 milliseconds.

49. A system for delivering a subcutaneous defibrillation therapy, comprising:
means for delivering, from a subcutaneous non-intrathoracic location relative to a patient's heart, a pre-shock waveform sufficient in energy such that contraction of skeletal musculature occurs in the patient's thorax but insufficient in energy for defibrillating the patient's heart; and
means for delivering, from the subcutaneous non-intrathoracic location or other subcutaneous non-intrathoracic location, a defibrillation waveform to the patient's heart during contraction of the skeletal musculature.

50. The system of claim 49, further comprising means for initiating a delay interval relative to delivery of the pre-shock waveform, wherein the defibrillation waveform is delivered following expiration of the delay interval.

51. The system of claim 50, wherein the delay interval has a pre-established duration.

52. The system of claim 50, wherein the delay interval has a duration of equal to or less than 2 seconds.

53. The system of claim 50, wherein the delay interval has a duration equal to or less than about 200 milliseconds.

54. The system of claim 50, wherein the delay interval has a duration equal to or less than about 100 milliseconds.

55. The system of claim 49, further comprising means for delivering the pre-shock waveform using a first vector and means for delivering the defibrillation waveform using the first vector.

56. The system of claim 49, further comprising means for delivering the pre-shock waveform using a first vector and means for delivering the defibrillation waveform using a second vector differing from the first vector.

57. The system of claim 49, further comprising means for detecting motion of the patient's thorax responsive to the pre-shock waveform, wherein the means for delivering the defibrillation waveform coordinates delivery of the defibrillation waveform in relation to the detected motion.
